(19) United States
(12) Patent Application Publication
(10) Pub. No.: US 2019/0015080 A1
(43) Pub. Date: Jan. 17, 2019

(54) Medical Diagnostic Ultrasound Imaging System and Method for Receiving Information from a Server During An Examination of a Patient to Improve Workflow

(71) Applicant: Imorgon Medical LLC, San Mateo, CA (US)

(72) Inventor: Lloyd B. Kreuzer, The Sea Ranch, CA (US)

(73) Assignee: Imorgon Medical LLC, San Mateo, CA (US)

(21) Appl. No.: 15/650,240

(22) Filed: Jul. 14, 2017

(51) Int. Cl. A61B 8/00; G01S 15/89; G06F 19/00; G06Q 50/24

(52) U.S. Cl. CPC A61B 8/585; A61B 8/00; G06F 19/327; G06F 19/322; G06Q 50/24; G01S 15/899

(57) ABSTRACT

A medical diagnostic ultrasound imaging system and method are provided for receiving information from a server during an examination of a patient to improve workflow. The information provided from the server can be information stored in a server-accessible storage location or generated by the server. The medical diagnostic ultrasound imaging system and server can have bi-directional communication to interact during the examination of the patient.

### BACKGROUND

A medical diagnostic ultrasound imaging system is used to conduct an ultrasound examination of a patient. Medical diagnostic ultrasound imaging systems are sometimes connected to a network, and information can be communicated to and from a server in the network before, during, or after the examination. For example, the medical diagnostic ultrasound imaging system can receive a list of scheduled patients from a study scheduling server before the examination, and the medical diagnostic ultrasound imaging system can store captured ultrasound images in an image server before, during, or after an examination.

### BRIEF DESCRIPTION OF THE DRAWINGS

FIG. 1 is a block diagram of a network environment of an embodiment.

FIG. 2 is a block diagram of a medical diagnostic ultrasound imaging system of an embodiment.

FIG. 3 is a block diagram of a server of an embodiment.

FIG. 4 is a screen shot of an exemplary DICOM modality worklist used to illustrate an embodiment.

FIG. 5 is a screen shot of an exemplary patient input screen of an embodiment.

FIG. 6 is an illustration of an ultrasound image of an embodiment.

FIG. 7 is an illustration of a patient worksheet of an embodiment.

FIG. 8 is a flow diagram of communications between a medical diagnostic ultrasound imaging system and a server of an embodiment.

FIG. 9 is a flow chart of a method of an embodiment for displaying a report of a prior study sent from a server to a medical diagnostic ultrasound imaging system during an examination of a patient.

FIG. 10 is a screen shot of an embodiment of a prior study list.

FIG. 11 is a flow chart of a method of an embodiment for displaying a drawing of a prior study sent from a server to a medical diagnostic ultrasound imaging system during an examination of a patient.

FIG. 12 is a flow chart of a method of an embodiment for displaying a patient worksheet sent from a server to a medical diagnostic ultrasound imaging system during an examination of a patient.

FIG. 13 is a screen shot of a display device of a medical diagnostic ultrasound imaging system of an embodiment in which an ultrasound image and a patient worksheet are simultaneously displayed on the display device.

FIG. 14 is an illustration of a medical diagnostic ultrasound imaging system of an embodiment in which an ultrasound image and a patient worksheet are simultaneously displayed on two separate display devices.

FIG. 15 is an illustration of an embodiment in which a server automatically generates a plot of current and previous measurements.

FIG. 16 is an illustration of a plot generated by a server of an embodiment of current and previous measurements.

FIG. 17 is an illustration of an embodiment in which a server returns data in response to a worksheet entry.

### DETAILED DESCRIPTION

By way of introduction, the following embodiments provide a medical diagnostic ultrasound imaging system and method for receiving information from a server during an examination of a patient to improve workflow. While some prior medical diagnostic ultrasound imaging systems can communicate with a server during an examination, such communication is very limited and does not provide the interactivity that the following embodiments can provide to improve workflow. For example, some prior medical diagnostic ultrasound imaging systems can send captured ultrasound images to a server and can receive an acknowledgment back from the server that the images were received and stored. In contrast, in one embodiment, in response to receiving a captured image or other information about a patient, the server can return information to the medical diagnostic ultrasound imaging system that can improve the sonographer's workflow in conducting the examination. That is, these embodiments can provide a simplified workflow because the sonographer gets information that she otherwise would not get. Also, by having the server provide an intelligent, processed result, these embodiments can make the sonographer perform different acts during the examination of the patient.

For example, if the medical diagnostic ultrasound imaging system sends the server a measurement of anatomy under study, and the server can look-up previous measurement(s) of the anatomy, generate a plot showing the current and previous measurements, and send the plot to the medical diagnostic ultrasound imaging system for display to the sonographer. This can help the sonographer decide which images or measurements to take next. As another example, as a sonographer is filling-out a patient study worksheet on the medical diagnostic ultrasound imaging system, the information completed on the worksheet can be sent the server. The server can process the information to determine what remaining sections of the worksheet are relevant and only return the relevant remaining sections to the medical diagnostic ultrasound imaging system. This can improve the sonographer's workflow by eliminating the need for the sonographer to page through irrelevant sections of the worksheet and can also guide the sonographer in which images and measurements to take during the rest of the examination. As another example, the medical diagnostic ultrasound imaging system can provide the server with information about the patient, and, in response to this information, the server can provide the medical diagnostic ultrasound imaging system with images or reports from a prior study of the patient, which the sonographer may find useful in conducting a present examination of the patient. As yet another example, the sonographer can initiate on the ultrasound system an instant message to the radiology reading room asking whether it is OK to release the current patient. The server can send this message (e.g., encrypted) to the active workstations in the radiology reading room. In one embodiment, any physician can “click” on the message, whereupon the server would remove the message from all other workstations and open on the “clicked” workstation the in-progress ultrasound exam showing the sonographer's study worksheet, flagged images, and a window capable of showing all images acquired thus far. The physician can review the images and other information and send an instant message back to the ultrasound machine that the patient can be released or, alternatively, that additional images, tests, or other actions are needed. Of course, these are just examples, and other implementations can be used. Some of these possible implementations are discussed below.

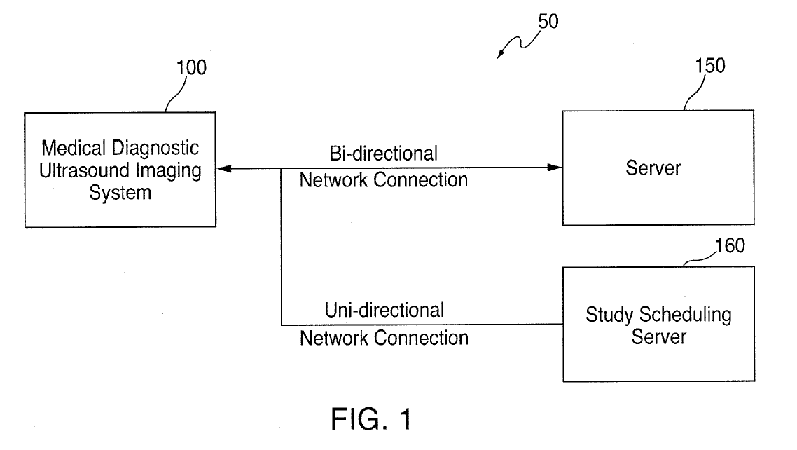

Turning now to the drawings, FIG. 1 is a block diagram of a network environment 50 of an embodiment. As shown in FIG. 1, the network environment 50 of this embodiment comprises a medical diagnostic ultrasound imaging system 100 (sometimes referred to herein as the “ultrasound system”), a server 150 in bi-directional communication with the medical diagnostic ultrasound imaging system 100, and a study scheduling server 160 in uni-directional communication with the medical diagnostic ultrasound imaging system 100. As used herein, “in communication with” could mean directly in communication with or indirectly (wired or wireless) in communication with through one or more components, which may or may not be shown or described herein. Also, the terms “study” and “examination” (or “exam”) will be used interchangeably herein.

As also used herein, the term “server” can mean a computer program or a device that provides functionality for other programs or devices (“clients”). Servers can provide various functionality (“services”), such as sharing data or resources among one or more clients or performing computation for one or more clients. A client can run on the same device as a server or may connect over a network to a server on a different device. Client-server systems can use a request-response communication technique in which a client sends a request to the server, and the server sends a response back to the client. In some embodiments, a server can be implemented as a physical server or a virtual server. Both can perform similar or identical functions, but their implementations can be different. For example, a physical server can be implemented using hardware (e.g., a CPU, memory, disk storage, network interface, etc), an operating system (e.g., Microsoft Windows Server 2012), and various software that may include, but is not limited to, drivers and application software. The term “physical server” can refer to the hardware, operating system, application, and other software. A virtual server can be implemented using hardware (e.g., a CPU, memory, disk storage, network interface, etc.), virtualizing software that acts to isolate the operating system from the hardware, an operating system, application, and other software. The term virtual server can refer to the operating system, application, and other software and exclude the hardware, or it can refer to both the software and the hardware. A virtual server may be located locally (e.g., in the medical institution's facilities) or “in the cloud” (e.g., when the virtual server is internet-based and provides shared processing resources and other on-demand services).

It should be noted that the network configuration 50 in FIG. 1 is merely one example, and other network configurations can be used. For example, in some embodiments, the study scheduling server 160 is not used or is in bi-directional communication with the medical diagnostic ultrasound imaging system 100. As another example, while FIG. 1 shows the communication between the medical diagnostic ultrasound imaging system 100, the server 150, and the study scheduling server 160 being over a network connection, in other embodiments, a network is not used (e.g., the medical diagnostic ultrasound imaging system 100 can be directly connected (not via a network) to the server 150 and/or the study scheduling server 160). Also, in another embodiment, the functionality of the server 150 and the study scheduling server 160 are combined into one device. It should also be understood that other devices (not shown) may be connected to the network and thereby be in bi-directional or uni-directional communication with the server 150. For example, various radiology workstations, radiology information systems (RIS), electronic medical record (EMR) systems, and PACS archives may be connected to the network.

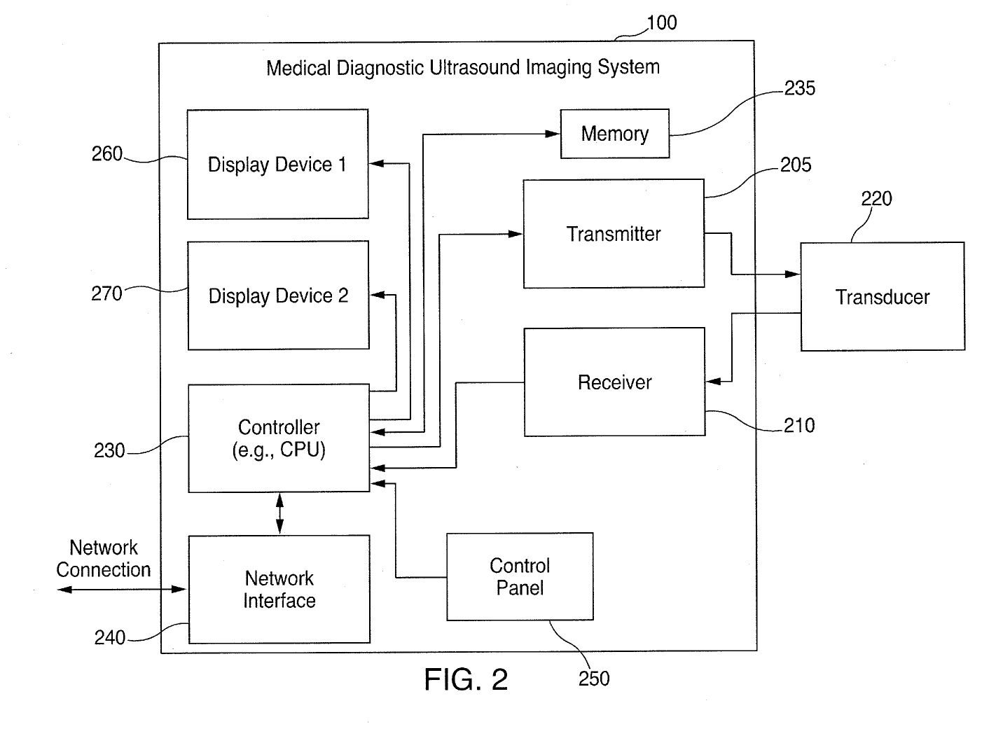

FIG. 2 is a block diagram of a medical diagnostic ultrasound imaging system 100 of an embodiment. In this embodiment, the medical diagnostic ultrasound imaging system 100 comprises at least one transmitter 205 and at least one receiver 210 in communication with a transducer 220 and a controller 230. The medical diagnostic ultrasound imaging system 100 also comprises a memory (one or more volatile and/or non-volatile devices) 235, a network interface 240, a control panel 250, and first and second display devices 260, 270. It should be noted that this is merely one implementation, and other implementations can be used.

In this embodiment, the controller 230 is configured to control the operation of the medical diagnostic ultrasound imaging system 100. The controller 230 can take the form, for example, of processing circuitry, a microprocessor or processor, logic gates, switches, an application specific integrated circuit (ASIC), a field programmable gate array, a programmable logic controller, an embedded microcontroller, a digital circuit, an analog circuit, combinations thereof, and other now-known or later-developed devices for analyzing and processing data. The controller 230 can be configured with hardware and/or firmware to perform the various functions described below and shown in the flow diagrams. The controller 230 can also execute software to perform these and other functions. For example, the memory 235 (e.g., a computer-readable medium) can store computer-readable program code (e.g., software) executable by a (micro)processor in the controller 230. The software can include a set of instructions that may be executed to cause the controller 230 to perform any one or more of the methods or computer-based functions disclosed herein.

In this embodiment, the control panel 250 includes one or more user input devices, such as, but not limited to, a keyboard, a number pad, real or virtual knobs and buttons, a joystick, a mouse or other pointing/selecting device, a touch screen, a camera, a microphone, and a remote control device. One or both of the display devices 260, 270 can take the form of a liquid crystal display (LCD), an organic light emitting diode (OLED), a flat panel display, a solid state display, a cathode ray tube (CRT), a projector, or other now-known or later-developed display device technology for outputting information. In one embodiment, one display device 260 is a relatively-larger display device, and the other display device 270 is a relatively-smaller display device with a touch-sensitive display. Also, instead of two display devices, the medical diagnostic ultrasound imaging system 100 can have one display device or more than two display devices.

The network interface 240, which may be part of the controller 230, can be created in software and/or may be a physical connection in hardware. The network interface 240 may be configured to connect with a network and/or external media. The connection with the network may be a physical connection, such as a wired Ethernet connection, and/or may be established wirelessly, such as when a wireless network is used (e.g., a cellular telephone network or an 802.11, 802.16, 802.20, or WiMax network). The network can be a public network, such as the Internet, a private network, such as an intranet, or combinations thereof, and may utilize a variety of networking protocols now available or later developed including, but not limited to, TCP/IP-based networking protocols. In one embodiment, the medical diagnostic ultrasound imaging system 100 acts as a client to the server 150 in a client-server network environment. However, in other embodiments, the medical diagnostic ultrasound imaging system 100 and the server 150 are peer computer systems in a peer-to-peer (or distributed) network environment. In one embodiment, the medical diagnostic ultrasound imaging system 100 and the server 150 communicate using the Digital Imaging and Communications in Medicine (DICOM) standard.

The transducer 220 can comprise an array of piezoelectric materials, which can take the form of, for example, a phased linear, linear, curved linear, sector, wide view, single element, 1.5 dimensional, two dimensional, or other type of array of transducer elements. The transducer 220 is configured to convert a waveform from the transmitter 205 to acoustic energy and convert received acoustic energy to electrical signals, which are provided to the receiver 210. The transmitter 205 can comprise digital and/or analog devices for generating the waveform along one or more scan lines, and the receiver 210 can comprise digital and/or analog devices for processing the electrical signals representing echo signals received by the transducer along the one or more scan lines, after the acoustic energy is reflected off of anatomy in the patient. The transmitter 205 and/or receiver 210 can comprise discrete analog circuitry, including amplifiers, filters, digital-to-analog converters, and analog-to-digital converters, for example.

In general, during a medical diagnostic ultrasound examination of a patient, a sonographer contacts the transducer 220 with the patient, and the medical diagnostic ultrasound imaging system 100 acquires an ultrasound image in accordance with settings configured in the controller 230 (e.g., as inputted via the control panel 250). In general, the controller 230 causes the transmitter 205 to apply a voltage to the transducer 220 to cause it to vibrate and emit an ultrasonic beam into the portion of the patient's body in contact with the transducer 220. Ultrasonic energy reflected from the patient's body impinges on the transducer 220, and the resulting voltages created by the transducer 220 are received by the receiver 210. The controller 230 processes the sensed voltages to create an ultrasound image and can display the image on one or both of the display devices 260, 270. In addition to being displayed, the ultrasound image can be stored in digital form on the medical diagnostic ultrasound imaging system 100 and/or the network. For example, the sonographer can be given the option of storing an ultrasound image by pressing an “image capture” key on the control panel 250, or, alternatively, the ultrasound image can be automatically stored without user intervention. In this way, a series of images from an ultrasound exam can be stored for later review and analysis.

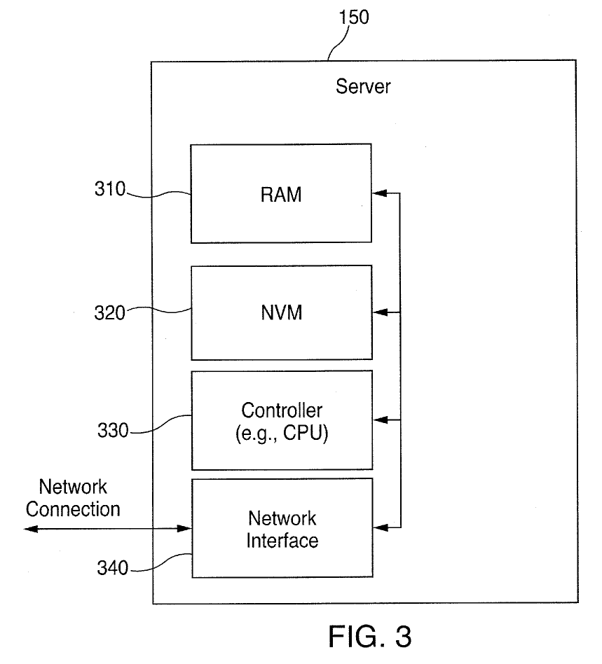

FIG. 3 is a block diagram of one implementation of the server 150 of an embodiment (other implementations can be used). As shown in FIG. 3, in this embodiment, the server 150 comprises RAM (or other volatile memory) 310, non-volatile memory 320 (e.g., solid state memory, a hard disk drive, an optical drive, etc.), a controller 330, and a network interface 340. The controller 330 and network interface 340 in the server 150 can take any suitable form, such as those described above with respect to the controller 230 and network interface 240, respectively, in the medical diagnostic ultrasound imaging system 100. However, the controller 330 and network interface 340 in the server 150 do not necessary need to be implemented in the same way as the controller 230 and network interface 240 in the medical diagnostic ultrasound imaging system 100. In this embodiment, the controller 330 in the server 150 is configured to implement the methods and algorithms discussed herein and shown in the accompanying flow charts to improve workflow in the medical diagnostic ultrasound imaging system 100.

Although not shown in the drawings, the study scheduling server 160 can be implemented in a similar way as the server 150. However, the controller in the study scheduling server 160 can be configured to provide study scheduling information to the medical diagnostic ultrasound imaging system 100. For example, the study scheduling server 160 can send a DICOM modality worklist to the medical diagnostic ultrasound imaging system 100 for display on the display device 260, 270. (While a worklist can be locally stored on the medical diagnostic ultrasound imaging system 100, having the study scheduling server 160 send a DICOM modality worklist to the medical diagnostic ultrasound imaging system 100 can provide department-wide uniformity.)

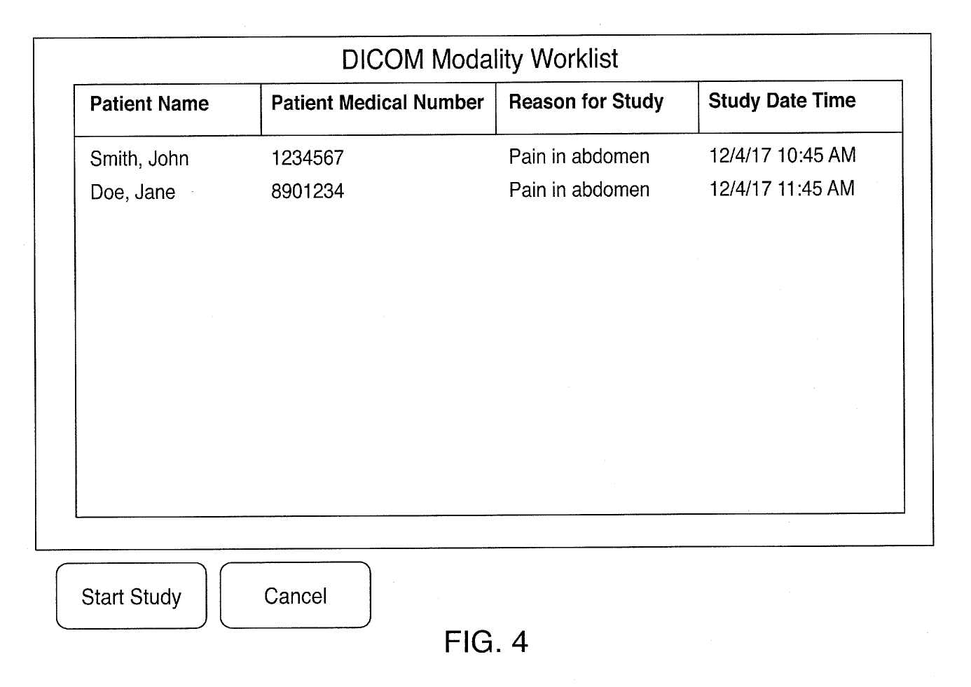

FIG. 4 is a screen shot of an exemplary DICOM modality worklist used to illustrate this embodiment. While a DICOM modality worklist is used in this example, it should be understood that other types of scheduled-patient lists or displays can be used. In general, a DICOM modality worklist is list of imaging procedures scheduled for the medical diagnostic ultrasound imaging system 100. As shown in FIG. 4, the DICOM modality worklist in this example includes patient name, patient medical number, reason for study, and study date and time. Other or different information can be displayed, such as, but not limited to, sex, age, the type of procedure, equipment type, procedure order, and referring physician. In operation, the sonographer selects the displayed line for the patient being examined and then presses a real or virtual “Start Study” button, which starts the ultrasound examination. These actions can be done using the control panel 250 (e.g., by using a mouse or cursor keys) or, if the display 260, 280 is touch-sensitive, but touching the displayed areas.

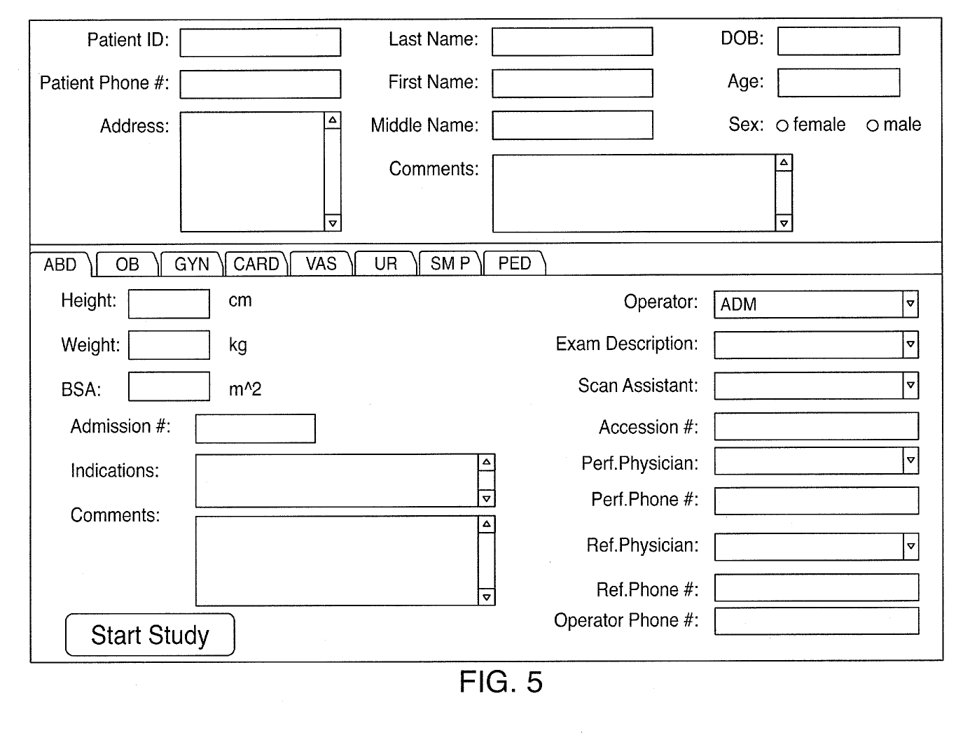

As an alternative to selecting a scheduled study from a DICOM modality worklist and then pressing the “Start Study” button, a sonographer can manually enter (e.g., using the control panel 250) information about a patient into a displayed form on the display device 260, 270. FIG. 5 is a screen shot of an exemplary patient input screen of an embodiment. As shown in FIG. 5, the sonographer can input information, such as patient ID and other patient-identifying information, as well as information about the examination. After the sonographer inputs the relevant information, the sonographer presses a real or virtual “Start Study” button, which starts the ultrasound examination.

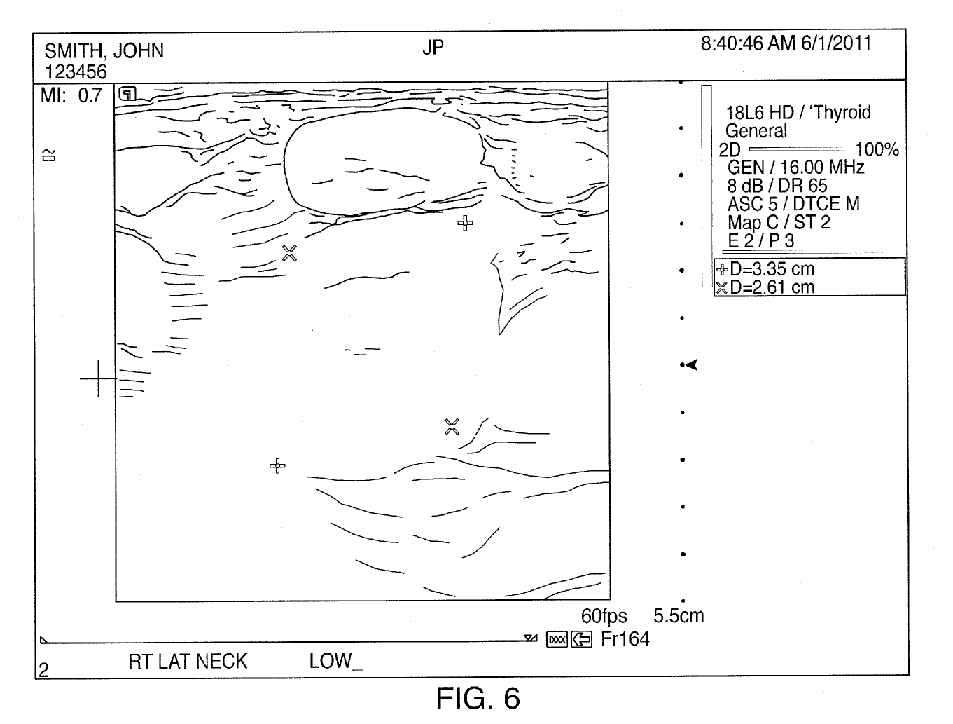

After the examination of the patient begins (e.g., after pressing the “Start Study” button on a DICOM modality worklist or a patient-input form), the sonographer conducts an ultrasound examination of the patient by contacting the transducer 220 to the patient and pressing a real or virtual “Capture” button on the medical diagnostic ultrasound imaging system 100 to generate ultrasound images (still images) or clips (i.e., a sequence of images that are played as a video (e.g., at 16 images/sec.)). FIG. 6 is an illustration of an example of an ultrasound image that can be displayed on the display device 260, 270 of the medical diagnostic ultrasound imaging system 100.

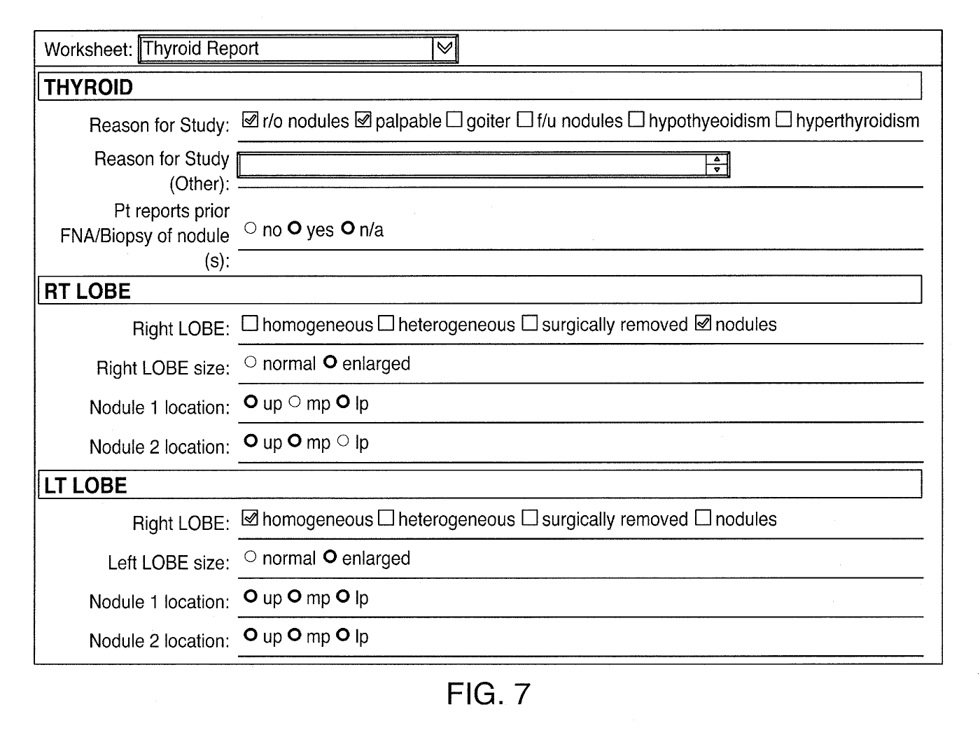

During the examination, the sonographer can also enter observations (e.g., using the control panel 250) into a worksheet (e.g., a patient study worksheet) displayed on the medical diagnostic ultrasound imaging system 100. FIG. 7 is an example of a worksheet for an ultrasound examination of a thyroid. The sonographer can enter observations by pointing and clicking to select check boxes and radio buttons and entering text in text boxes.

After the sonographer captures all of the needed ultrasound images and completes the appropriate worksheet(s), the sonographer ends the examination by pressing a real or virtual “End Study” button. When the examination ends, the medical diagnostic ultrasound imaging system 100 can store the captured images, completed worksheet, and other information in the medical diagnostic ultrasound imaging system 100 and/or in the network (e.g., in the server 150) or in a storage location connected to the network such as a PACS archive.

During the time interval beginning when the “Start Study” button is pressed (or the equivalent) and when the “End Study” button is pressed (or the equivalent), the ultrasound exam may be described as “in progress.” In one embodiment, the ultrasound machine is programmed to send in-progress captured images, measurements, and other information to the server immediately after these items are generated rather than waiting until the entire ultrasound exam is completed to send these items. When the ultrasound machine sends these data and various sonographer inputs to the server while the exam is in progress, the server can provide useful in-progress feedback to the ultrasound machine for use by the sonographer to improve workflow and facilitate the ultrasound study. In other embodiments, the ultrasound machine sends its information to the server after a “Pause Study” button, or the equivalent, is pressed, and the server returns information or other feedback to the ultrasound machine for the sonographer. In these embodiments, the sonographer may need to “Un-Pause,” re-open, or re-start the study in order to perform additional measurements or additional imaging of the patient based on the feedback provided by the server. These other embodiments may provide a less-efficient workflow, but they may allow the ultrasound machine to “batch send” images and data rather than continuously send the images and data as they are acquired. For all of the above embodiments, the communication between the ultrasound machine and the server can be defined as occurring “during an examination” because the patient remains in the ultrasound exam room and the sonographer has the option of adding additional images, measurements, or other data to the patient's ultrasound exam.

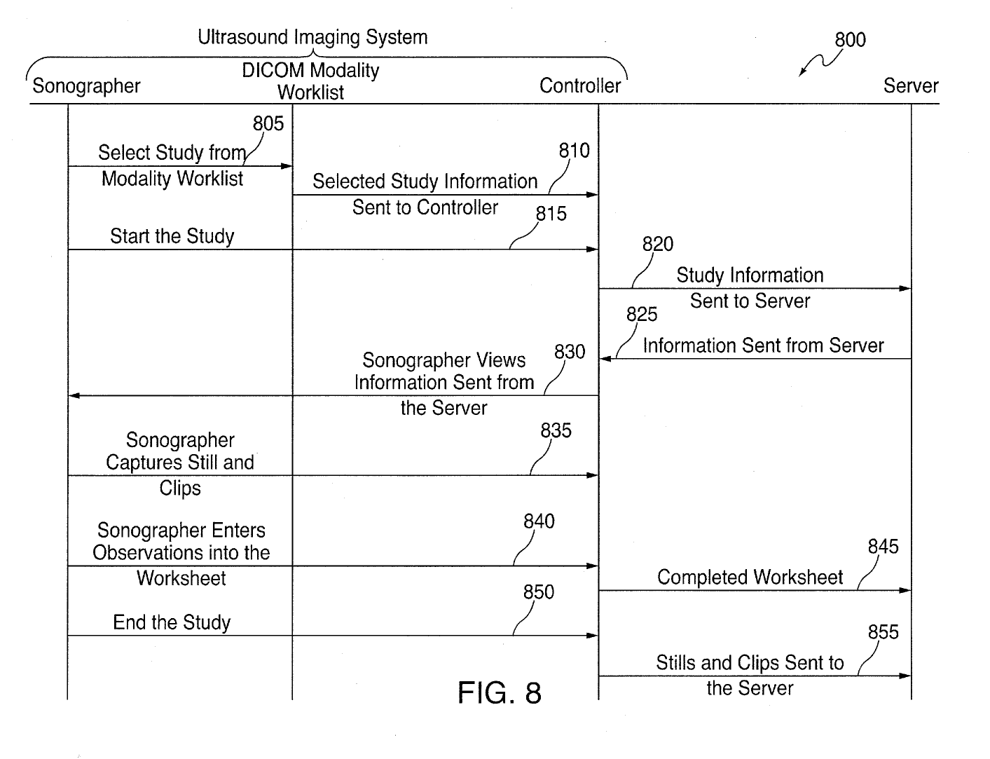

FIG. 8 is a flow diagram 800 of communication between the medical diagnostic ultrasound imaging system 100 and server 150. This is merely an example, and other flows are possible. Many of these acts are similar to those discussed above in conjunction with the normal operation of an ultrasound system (e.g., selecting a study from a modality worklist sent from a study scheduling server (act 805), sending the selected study information to the controller in the ultrasound system (act 810), starting the study/examination (act 815), capturing images (act 835), entering observations into a worksheet (act 840), and sending images to a server after the study has ended (acts 850 and 860).

While some prior medical diagnostic ultrasound imaging systems can communicate with a server during an examination, such communication is very limited. For example, some prior medical diagnostic ultrasound imaging systems can send captured ultrasound images to a server and can receive an acknowledgment back from the server that the images were received and stored. In contrast, the following embodiments provide a level of interactivity between the medical diagnostic ultrasound imaging system and the server that can improve workflow during the examination.

More specifically, in this embodiment, the medical diagnostic ultrasound imaging system 100 sends first information about the patient to the server 150 (act 820). The first information can be sent in an encrypted or unencrypted (“in the clear”) form. The “first information about the patient” can take any suitable form and can serve as a way for the server 150 to locate relevant information to send back to the medical diagnostic ultrasound imaging system 100 during the examination and/or can be processed by the server to provide a unique result to the medical diagnostic ultrasound imaging system 100. For example, the “first information about the patient” can be patient-identifying information, such as the patient's name or identifier, as selected from a DICOM modality worklist (see FIG. 4) or as inputted by a sonographer on a patient-input form (see FIG. 5). As another example, the “first information about the patient” can be something other than or in addition to information that merely identifies the patient. For example, “first information about the patient” can be a protocol step taken in a diagnostic procedure of a patient, information about the patient's study (e.g., a designation of the type of study being conducted on the patient (such as study type=abdomen or study type=leg), a unique study identifier for the patient's study, or accession number associated with the patient), or information about the patient that is generated during the examination of the patient (e.g., a captured image or measurement of an anatomy of the patient). The term “patient information” will be used herein to refer to any information about the patient, including information identifying the patient and information derived from an examination of the patient.

While patient identifier and/or a study identifier will be used in this example to illustrate these embodiments, it should be noted that other forms of “first information the patient” can be used. For example, the first information sent to the server 150 comprises information entered into a patient worksheet, which can be found locally on the medical diagnostic ultrasound imaging system 100 or sent from the server 150 (e.g., the server 150 can select which patient worksheet to send to the medical diagnostic ultrasound imaging system 100 based on the patient identifier and/or study identifier). The information entered into the patient worksheet can take any form, including, but not limited to, a keystroke input, a mouse click input, a selection of a cursor location, a selection of a touch screen location, a measured value, a calculated value, an identification of a value, information about the examination, and information about a protocol step in a diagnostic procedure. As another example, the first information about the patient sent to the server 150 includes one or more of the following: a medical diagnostic ultrasound image, a reference to a medical diagnostic ultrasound image, an “instant message” created by the sonographer, and a sequence of medical diagnostic ultrasound images. As shown from these examples, “first information about the patient” can be information that is derived from the current examination of the patient. Examples of information that is derived from an examination of a patient include, but are not limited to, a medical diagnostic ultrasound image of the patient, a reference to a medical diagnostic ultrasound image of the patient, a sequence (a “clip”) of medical diagnostic ultrasound images of the patient, and a measurement, observation, or finding entered into a worksheet on the medical diagnostic ultrasound imaging system. Of course, these are merely examples, and other information about the patient can be sent to the server 150.

The first information can be sent to the server 150 before or after the examination begins or at the exact moment the examination begins. For example, when the first information about the patient is a patient identifier and/or a study identifier obtained from a DICOM modality worklist, the first information about the patient can be send before or after the “Start Study” button is pressed, or simultaneously when the “Start Study” button is pressed. Also, as will be discussed in more detail below, “first information” is not necessarily the first data transmission from the medical diagnostic ultrasound imaging system 100 and the server 150. Further, there can be multiple instances of “first information” sent from the medical diagnostic ultrasound imaging system 100 to the server 150 (and multiple instances of “second information” returned from the server 150 in response) during an examination to provide multiple instances of interactivity.

When the server 150 receives the first information about the patient from the medical diagnostic ultrasound imaging system 100, the controller 330 in the server 150 uses the first information to locate or generate “second information” about the patient. For example, the non-volatile memory 320 of the server 150 can store various information about patients in a database or other data structure, and the controller 330 in the server 150 can query the database with the first information about the patient (e.g., the patient identifier and/or study identifier) to locate the relevant second information about the patient. Instead of being stored in the non-volatile memory 320 of the server 150, the second information can be stored in an external storage location, such as an electronic medical record (EMR) system or archive and a picture archive and communication system (PACS) archive. Accordingly, the second information can be accessed by the server 150 from a local or external storage location. As another example, if the first information is a measurement of an anatomy of the patient, the server 150 can process the information to return a chart of current and previous measurements of the anatomy. As yet another example, if the first information is information entered into a patient study worksheet, the server 150 can process the entered worksheet information to generate information customized for the specific protocol being used to guide the sonographer to perform certain steps.

Referring back to FIG. 8, after the server 150 locates or generates the second information about the patient based on the first information about the patient, the server 150 sends the second information to the medical diagnostic ultrasound imaging system 100 (act 825). As with the first information, the second information can be sent in an encrypted or unenerypted (“in the clear”) form. Also, the first and second information can both be encrypted or both be unencrypted, or one of the first and second information can be encrypted, while the other of the first and second information can be unencrypted. This information can be sent in any suitable way. For example, in one embodiment, the server 150 streams the second information to the medical diagnostic ultrasound imaging system 100. As used herein, “streaming” refers to the process that allows part of a file to be displayed after is it received, instead of having to wait for the entire file to be downloaded before any part of the file can be displayed.

It should be noted that while FIG. 1 shows a bi-directional connection between the medical diagnostic ultrasound imaging system 100 and the server 150, there can by bi-directional communication or uni-directional communication during an examination. For example, if the first information about the patient is sent to the server 150 during the examination and the second information about the patient is received from the server 150 during the examination, there would be bi-directional communication during the examination. However, if the first information about the patient is sent to the server 150 before the start of the examination, there may only be uni-directional communication during the examination, with the server 150 sending the second information about the patient. That being said, bi-directional communication during the examination allows multiple instances of interactivity during the examination that may be useful in certain situations to improve workflow.

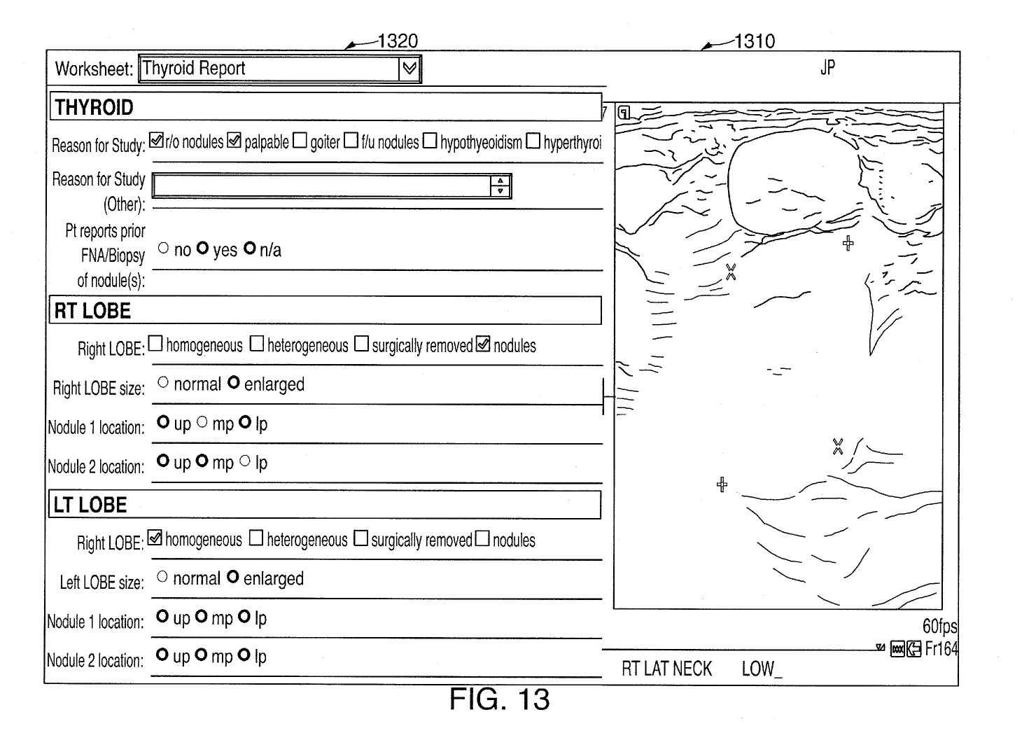

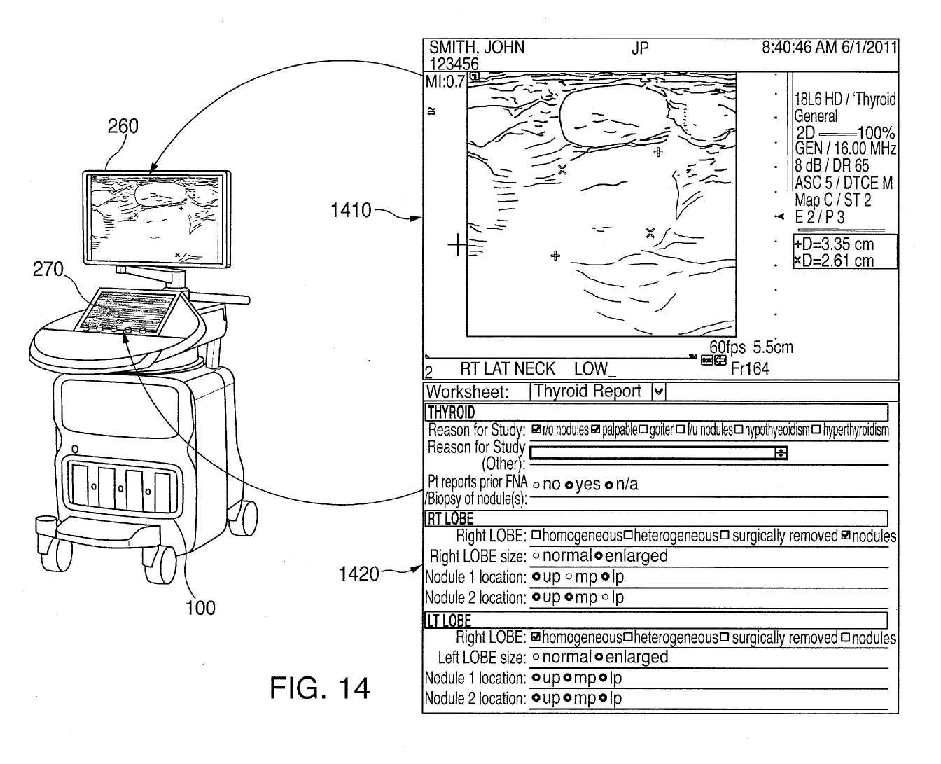

After the medical diagnostic ultrasound imaging system 100 receives the second information about the patient from the server 150, the sonographer can view the second information on one or both of the display devices 260, 270 of the medical diagnostic ultrasound imaging system 100 (act 830). If the medical diagnostic ultrasound imaging system 100 has more than two display devices, the second information can be displayed on the additional display devices as well. For example, the second information about the patient received from the server 150 can be simultaneously displayed (e.g., on one or more display devices) with a medical diagnostic ultrasound image generated by the medical diagnostic ultrasound imaging system 100. For example, as shown in FIG. 13, an ultrasound image (e.g., from a current study) 1310 can be simultaneously displayed with a patient worksheet 1320 on one of the display devices 260, 270 of the medical diagnostic ultrasound imaging system 100. Alternatively, as shown in FIG. 14, the ultrasound image 1410 and the patient worksheet 1420 can be displayed simultaneously, with the ultrasound image 1410 being displayed on the first display device 260 at the same time the patient worksheet 1420 is displayed on the second display device 270.

The “second information about the patient” can take any suitable form. In one embodiment, the “second information about the patient” is information for use by a sonographer during the examination of the patient. Examples of “second information about the patient” include, but are not limited to, an automatically-selected medical diagnostic ultrasound image of the patient from a prior examination, a drawing made during a prior examination, a prompt to acquire an additional medical diagnostic ultrasound image of the patient, an instant message from a radiologist, a processed image from a current examination of the patient (e.g., such as when artificial intelligence is used to identify and grade a lesion or perform another type of analysis), information from the patient's medical record, the name of the doctor who ordered a prior study, the reason for the prior study, a reading physician's report to the referring physician of the prior study, measured values from the prior study, and trended values of the prior study. As another example, the second information about the patient can be a patient study worksheet tailored to the patient. For example, as a sonographer is filling-out a patient study worksheet on the medical diagnostic ultrasound imaging system, the information completed on the worksheet (the “first information”) can be sent to the server 150. The server 150 can process the first information to determine what remaining sections of the worksheet are relevant and only return the relevant remaining sections (the “second information”) to the medical diagnostic ultrasound imaging system 100. Of course, these are just examples, and other information can be provided.

It should be noted that, in some embodiments, the “second information about the patient” is sent after the server 150 sends an acknowledgement of receipt of the “first information about the patient” or some other transmission further to a communications protocol. That is, in some embodiments, the “second information about the patient” is information identified by or generated from the “first information about the patient” and excludes ancillary communication protocol transmissions.

The following paragraphs provide examples of different types of “second information about a patient” received from the server 150. It should be noted that these are merely examples and that other implementations can be used.

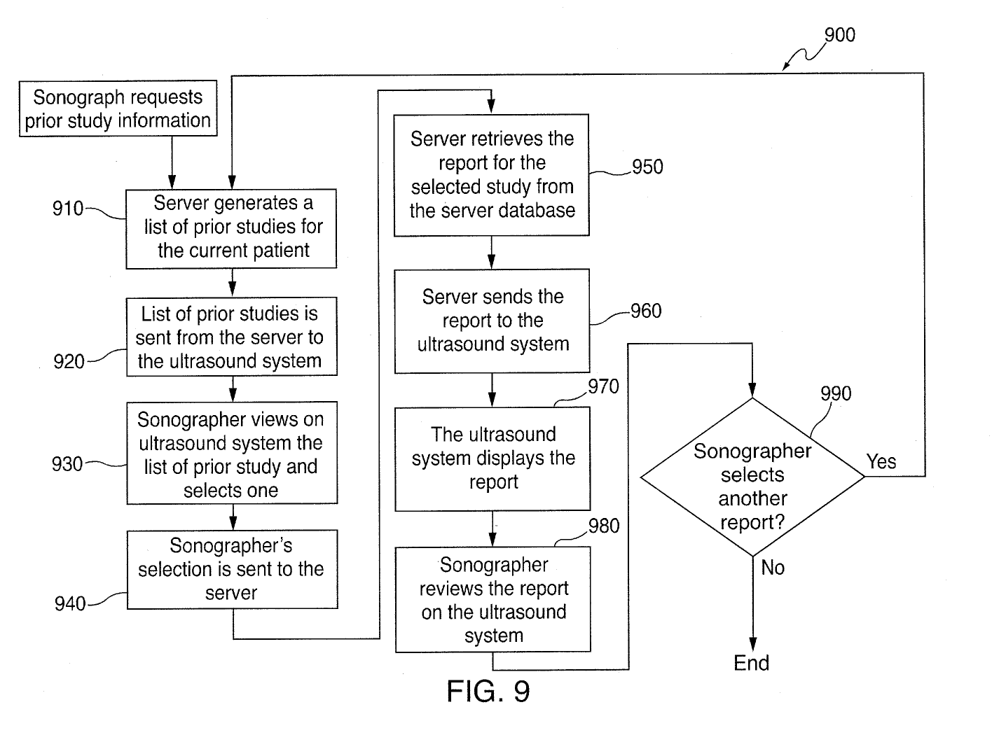

Returning to the drawings, FIG. 9 is a flow chart 900 that illustrates a situation in which the “second information about a patient” received from the server is a report of a prior study. A prior study can be useful to the sonographer in conducting the current examination, as it can suggest to the sonographer what images and/or measurements to take. As shown in FIG. 9, in response to the server 150 receiving the first information about the patient from the medical diagnostic ultrasound imaging system 100, the server 150 generates a list of prior studies for the current patient (act 910) and sends the list to the medical diagnostic ultrasound imaging system 100 (act 920). The sonographer then views the list of prior studies on one of the displays 260, 270 and selects a desired study and function button (act 930). The selection is then sent to the server 150 (act 940).

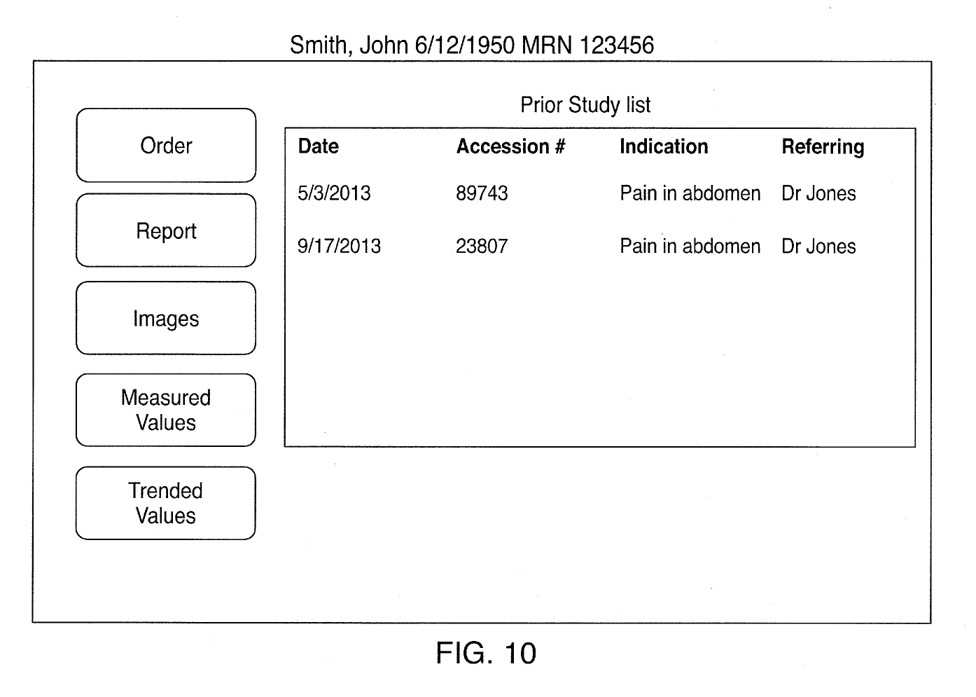

FIG. 10 is an example of a screen shot of prior study list sent from the server 150 and displayed on the medical diagnostic ultrasound imaging system 100. As shown in FIG. 10, in this example, the prior study list for this patient include two prior studies, identified by date, accession number, indication, and referring physician. The sonographer can select a desired study by using the control panel 250 (e.g., a mouse or keyboard). In this example, the sonographer has five options to view information from a selected study: Order, Report, Images, Measured Values, and Trended Values. The sonographer can select one of these options by clicking on the desired displayed button. Selecting the order button will result in the display of information about the order for the selected prior study (e.g., the name of the doctor who ordered the study and the reason for the study). Selecting the report button will result in the display of a report generated for the study (e.g., the reading physician's report to the referring physician). Selecting the images button will result in the display of ultrasound images from the selected prior study. Selecting the measured values button will result in the display of values measured for the selected study (e.g., for a thyroid study, the length of a thyroid nodule). Selecting the trended values button will result in the display of a plot of measured values over time (e.g., for a thyroid study, a plot of the length of the nodule over time).

In this example, the selected function was “Report,” so the server 150 retrieves the report for the selected study from the server's database (e.g., stored in RAM 310 or non-volatile memory 320) (act 950). The server 150 then sends the report to the medical diagnostic ultrasound imaging system 100 (act 960), and the medical diagnostic ultrasound imaging system 100 displays the report (act 970). The sonographer then can review the report on the medical diagnostic ultrasound imaging system 100 (act 980) and select another report, if desired (act 990). Flows similar to the one shown in FIG. 9 would take place if the sonographer presses the Order, Images, Measured Values, or Trended Values buttons.

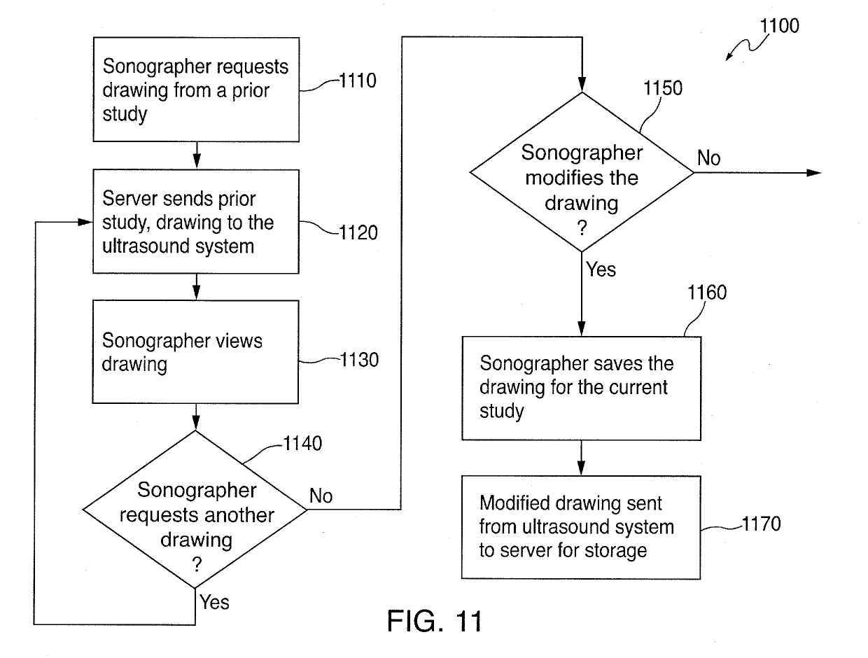

It should be noted that the buttons shown on the screen shot in FIG. 10 are just examples, and other buttons can be used. For example, in another embodiment, the sonographer has the option of pushing a Drawing button. A flow chart 1100 illustrating this method is presented in FIG. 11. As shown in FIG. 11, when the sonographer clicks on the Drawing button (act 1110), a signal is sent to the server 150, and the server 150 sends a drawing from a prior study to the medical diagnostic ultrasound imaging system 100 (act 1120). The sonographer can then view the drawing from the selected prior study on the display 260, 270 of the medical diagnostic ultrasound imaging system 100 (act 1120). The drawing from the prior study can be a sketch the prior sonographer or reading physician made of some anatomy of the patient (e.g., the shape of a tumor or the approximate location of a tumor within an organ). There can be several prior drawings made, and the sonographer has the option of requesting additional drawings, if needed (act 1140). If the sonographer sees a change in the anatomy with respect to what is shown in the drawing from the prior study, the sonographer can create a modified version of the drawing (e.g., showing the new shape of the anatomy). In such a situation (act 1150), the sonographer can save the new drawing for the current study locally on the medical diagnostic ultrasound imaging system 100 (act 1160). The new drawing can also be sent from medical diagnostic ultrasound imaging system 100 to the server 150 for storage (act 1170).

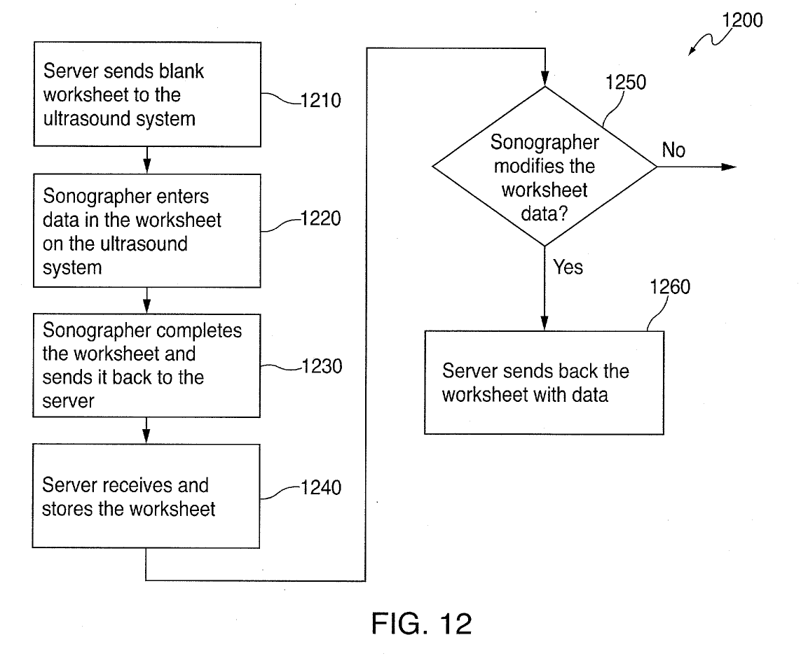

In addition to providing information about prior data about the patient, the server 150 can provide the medical diagnostic ultrasound imaging system 100 with the patient worksheet based on the first information about the patient sent to the server 150. This example will now be illustrated in conjunction with the flow chart 1200 in FIG. 12. As shown in FIG. 12, in response to receiving first information about the patient from the medical diagnostic ultrasound imaging system 100, the server 150 sends a blank patient worksheet (such as the one shown in FIG. 7) to the medical diagnostic ultrasound imaging system 100 (act 1210). The sonographer can then enter data into the worksheet using the control panel 250 of the medical diagnostic ultrasound imaging system 100 (act 1220). For example, the information entered into the patient worksheet can comprise one or more of the following: a keystroke input, a mouse click input, a selection of a cursor location, a selection of a touch screen location, a measured value, a calculated value, an identification of a value, information about the examination, and information about a protocol step in a diagnostic procedure. After the sonographer completes the worksheet, it is sent back to the server 150 (act 1230), which stores the worksheet in its memory (act 1240). If the sonographer later wants to modify the worksheet data (act 1250), the server 150 can send the stored worksheet (with previously-entered data) back to the medical diagnostic ultrasound imaging system 100 (acts 855 (FIG. 8) and 1260 (FIG. 12)). While FIG. 8 shows the completed worksheet sent to the server 150 before the end of the study, in other embodiments, the completed worksheet can be sent after the end of the study. Also, while FIG. 8 shows the ultrasound images sent to the server after the end of the study, in other embodiments, the ultrasound images can be sent before the end of the study.

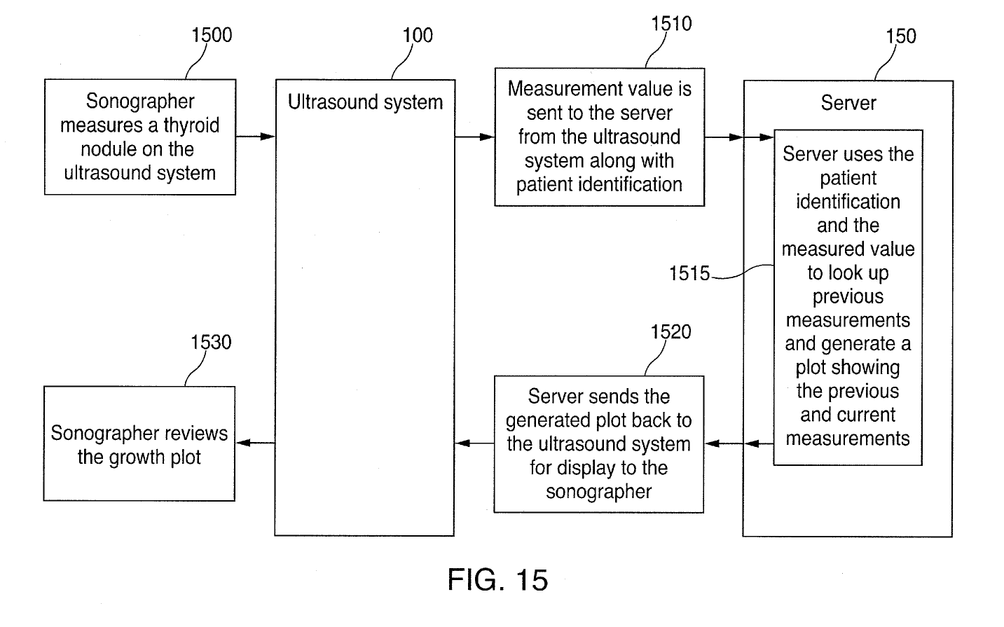

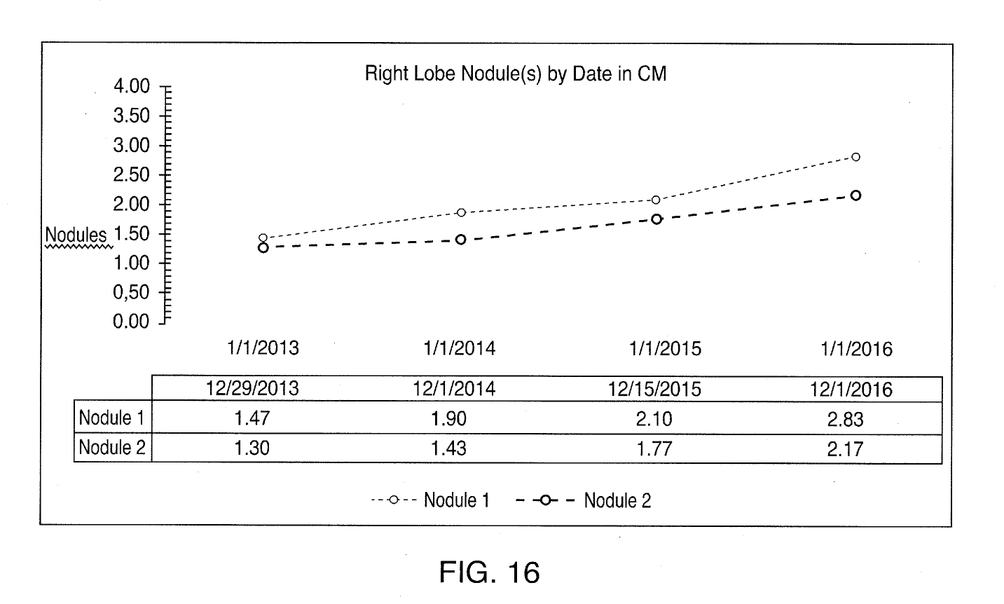

While some of the above examples show the server 150 using the first information to look-up stored second information, as noted above, the server 150 can additionally or alternatively process the first information to generate the second information (e.g., the server 150 can provide information that is “unique,” in that it is not previously stored in either the medical diagnostic ultrasound imaging system 100 or the server 150). For example, if the medical diagnostic ultrasound imaging system 100 sends the server 150 a measurement of anatomy under study, and the server 150 can look-up previous measurement(s) of the anatomy, generate a plot showing the current and previous measurements, and send the plot to the medical diagnostic ultrasound imaging system 100 for display to the sonographer. By generating a plot that did not exist before, the server 150 is doing something more than just looking up and returning stored data. That is, the server 150 responds to information about the patient (here, information that is provided by the sonographer and was derived during the current examination of the patient) by processing the information and sending back new information to the ultrasound system 100. This example is illustrated in FIGS. 15 and 16. In FIG. 15, the sonographer measures a thyroid nodule of a patient on the medical diagnostic ultrasound imaging system 100 (act 1500). The measurement value, along with the patient identification, is sent to the server 150 (act 1510). The server 150 uses the patient identification and the measured value to look-up previous measurements of the patient's thyroid and automatically generates a plot (see FIG. 16) showing the previous and current measurements (act 1515). The server 150 then sends the generated plot back to the medical diagnostic ultrasound imaging system 100 for display to the sonographer (act 1520). The sonographer can then review the growth plot (act 1530), which can assist the sonographer in the current study.

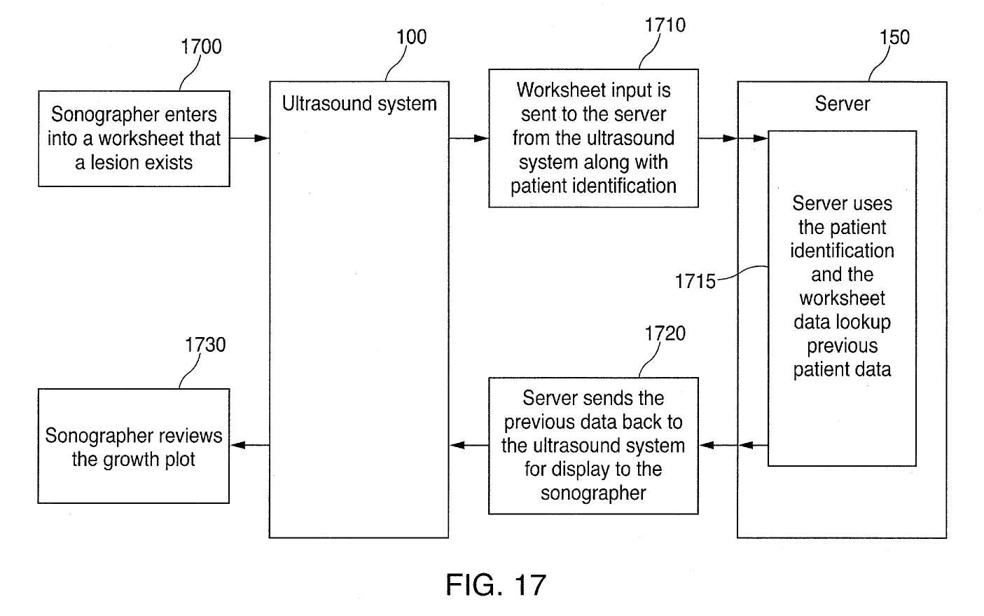

As another example, as a sonographer is filling-out a patient study worksheet on the medical diagnostic ultrasound imaging system 100, the information completed on the worksheet can be sent to the server 150. The server 150 can process the information to determine what remaining sections of the worksheet are relevant and only return the relevant remaining sections to the medical diagnostic ultrasound imaging system 100. As a related additional example, consider a situation in which the sonographer indicates on a patient study worksheet that there is a cyst on the patient's liver, and that information is sent to the server 150. In response to this information, the server 150 can modify the patient study worksheet to include fields asking for the diameter of the cyst and whether the cyst is clear or contains debris. By receiving this modified worksheet on the ultrasound system 100, the sonographer can be guided on next steps during the examination. As yet another example (see FIG. 17), after the sonographer enters into a worksheet that a lesion exists (act 1700), the worksheet input, along with the patient identification, is sent to the server 150 (act 1710). The server 150 uses the patient identification and the worksheet data to look-up previous patient data (act 1715) and returns it for display and use by the sonographer (acts 1720 and 1730).

Also, while each diagram discussed above may show a single instance of “first information” sent from the medical diagnostic ultrasound imaging system 100 and a single instance of “second information” sent from the server 150, as noted above, there can be multiple sequences of “first information” sent from the medical diagnostic ultrasound imaging system 100 and multiple sequences of corresponding “second information” sent from the server 150. This provides multiple instances of interactivity that can improve workflow. Also, the various instances of “first information” can be sent at different points during the examination. For example, patient identification information can be sent to the server 150 at the very start of the examination, whereas a measure of anatomy can be sent to the server 150 at some point after the start of the examination.

It should also be noted that, in one embodiment, the functionality of interacting with the server 150 is performed by a controller 230 integrated in the medical diagnostic ultrasound imaging system 100. As such, the functionality of interacting with the server 150 is portable with the medical diagnostic ultrasound imaging system 100 and does not require an additional computer local to the medical diagnostic ultrasound imaging system 100 (e.g. a separate network-connected computer in the examination room). Also, although the medical diagnostic ultrasound imaging system 100 interacts with the server 150, it is more than just a substitute for a computer terminal, as a computer terminal does not both generate ultrasound images and accept sonographer measurements made using electronic calipers on the ultrasound machine, sonographer observations, etc. That is, having one system being used to both conduct the examination and interact with the server provides advantages over using two separate devices.

There are many alternatives and additions that can be used with these embodiments. For example, there can be instant messaging between the operator of the medical diagnostic ultrasound imaging system and a physician or other person, which can provide an efficient communication mechanism. For example, the operator can enter a message into the medical diagnostic ultrasound imaging system, and the medical diagnostic ultrasound imaging system can send the message to a physician or other person (e.g., via the server or via another communication channel). The message can include text entered by the operator and/or other information, such as, but not limited to, a reference to a patient, a reference to a study, a reference to one or a few medical images previously “tagged” by the sonographer for comment by the physician, or a reference to the entire exam up to this point in time. The physician or other person receiving the message can then send a response back to the operator (e.g., via the server or via another communication channel) that can provide direction or guidance to the sonographer on how to complete the exam.

Finally, although a medical diagnostic ultrasound imaging system was used above as an example to illustrate these embodiments, it should be noted that these embodiments can be implemented in other medical diagnostic imaging system. Examples of other imaging modalities include, but are not limited to, computed tomography (CT), magnetic resonance imaging (MRI), computed radiography, magnetic resonance, angioscopy, color flow Doppler, cystoscopy, diaphanography, echocardiography, fluoresosin angiography, laparoscopy, magnetic resonance angiography, positron emission tomography, single-photon emission computed tomography, x-ray angiography, computed tomography, nuclear medicine, biomagnetic imaging, culposcopy, duplex Doppler, digital microscopy, endoscopy, fundoscopy, laser surface scan, magnetic resonance spectroscopy, radiographic imaging, thermography, and radio fluroscopy. Further, the phrase “medical diagnostic” is used herein to distinguish from “therapeutic” systems (e.g., ultrasound system that uses ultrasonic waves to destroy kidney stones). However, it should be noted that these embodiments can be used in therapeutic systems as well.

It is intended that the foregoing detailed description be understood as an illustration of selected forms that the invention can take and not as a definition of the invention. It is only the following claims, including all equivalents, that are intended to define the scope of the claimed invention. Finally, it should be noted that any aspect of any of the preferred embodiments described herein can be used alone or in combination with one another.

1. A medical diagnostic ultrasound imaging system with an improved graphical user interface that dynamically displays a worksheet, the medical diagnostic ultrasound imaging system comprising:
at least one transmitter;
at least one receiver;
a set of user input devices;
at least one display device; and
a controller in communication with the at least one transmitter, the at least one receiver, the set of user input devices, and the at least one display device, wherein the controller is configured to perform the following after receiving a command signaling a beginning of an examination of a patient but before receiving a command signaling an end of the examination of the patient:
display on the at least one display device an ultrasound image of the patient, wherein the ultrasound image of the patient is generated by the medical diagnostic ultrasound imaging system; and
dynamically display a worksheet in a graphical user interface on a same or different one of the at least one display device that displays the ultrasound image of the patient by performing the following, wherein the worksheet comprises a plurality of fields with multiple choice options for sonographer selection, wherein the multiple choice options specify different observations of the ultrasound image of the patient:
display a first section of the worksheet in the graphical user interface;
receive a selection of one of the multiple choice options in the worksheet;
send the selected multiple choice option to a server, wherein the server stores a plurality of additional sections of the worksheet, some of which are associated with the selected multiple choice option and some of which are not associated with the selected multiple choice option, and wherein the server is configured to identify an additional section of the worksheet that is associated with the selected multiple choice option;
receive, from the server, the additional section of the worksheet that is associated with the selected multiple choice option, wherein sections of the worksheet that are not associated with the selected multiple choice option are not received from the server; and
display the additional section of the worksheet that is associated with the selected multiple choice option in the graphical user interface;
wherein the sending of the selected multiple choice option to the server and the receiving and displaying of the additional section of the worksheet that is associated with the selected multiple choice option occur during the examination of the patient, and wherein such interactivity between the medical diagnostic ultrasound imaging system and the server improves workflow during the examination of the patient by avoiding display of sections of the worksheet in the graphical user interface that are not associated with the selected multiple choice option;
wherein the medical diagnostic ultrasound imaging system uses the set of user input devices to receive both a setting used to generate the ultrasound image of the patient and the selection of the multiple choice option.

2. The medical diagnostic ultrasound imaging system of claim 1, wherein the ultrasound image of the patient and the worksheet are simultaneously displayed.

3. The medical diagnostic ultrasound imaging system of claim 1, wherein only one of ultrasound image of the patient and the worksheet is displayed at any given time.

4. The medical diagnostic ultrasound imaging system of claim 1, wherein after receiving the command signaling the beginning of the examination of the patient but before receiving the command signaling the end of the examination of the patient, the controller is further configured to interact with the server by sending first information about the patient to the server and, in response, receiving additional information about the patient from the server.

5. The medical diagnostic ultrasound imaging system of claim 4, wherein the first information about the patient comprises at least one of a patient identifier and a study identifier.

6. The medical diagnostic ultrasound imaging system of claim 4, wherein the first information about the patient is derived from the examination of the patient.

7. The medical diagnostic ultrasound imaging system of claim 4, wherein the additional information about the patient is not specifically requested by the medical diagnostic ultrasound imaging system, and wherein the server is configured to determine what additional information about the patient to send based on the first information about the patient received from the medical diagnostic ultrasound imaging system.

8. The medical diagnostic ultrasound imaging system of claim 4, wherein the additional information about the patient is stored in a server-accessible storage location prior to the medical diagnostic ultrasound imaging system sending the first information about the patient to the server.

9. The medical diagnostic ultrasound imaging system of claim 4, wherein the additional information about the patient is generated by the server after the medical diagnostic ultrasound imaging system sends the first information about the patient to the server.

10. The medical diagnostic ultrasound imaging system of claim 1, wherein the controller is further configured to
receive second sonographer input in the additional section of the worksheet;
send the second sonographer input to the server;
receive, from the server, yet another section of the worksheet; and
display the yet another section of the worksheet received from the server in the graphical user interface.

11. The medical diagnostic ultrasound imaging system of claim 1, wherein the controller is integrated in the medical diagnostic ultrasound imaging system.

12. The medical diagnostic ultrasound imaging system of claim 1, wherein the controller is further configured to send and receive instant messages.

13. A method for improving workflow during an examination of a patient using a medical diagnostic ultrasound imaging system with an improved graphical user interface that dynamically displays a worksheet, the method comprising:
performing the following in a medical diagnostic ultrasound imaging system during an ultrasound examination of a patient:
displaying an ultrasound image of the patient on a display device of the medical diagnostic ultrasound imaging system, wherein the ultrasound image of the patient is generated by the medical diagnostic ultrasound imaging system; and
dynamically displaying a worksheet in a graphical user interface on a same or different display device that displays the ultrasound image of the patient by performing the following, wherein the worksheet comprises a plurality of fields with multiple choice options for sonographer selection, wherein the multiple choice options specify different observations of the ultrasound image of the patient:
displaying a first section of the worksheet in the graphical user interface;
receiving a selection of one of the multiple choice options in the worksheet,
sending the selected multiple choice option to a server, wherein the server stores a plurality of additional sections of the worksheet, some of which are associated with the selected multiple choice option and some of which are not associated with the selected multiple choice option, and wherein the server is configured to identify an additional section of the worksheet that is associated with the selected multiple choice option;
receiving, from the server, the additional section of the worksheet that is associated with the selected multiple choice option, wherein sections of the worksheet that are not associated with the selected multiple choice option are not received from the server; and
displaying the additional section of the worksheet that is associated with the selected multiple choice option in the graphical user interface;
wherein such interactivity between the medical diagnostic ultrasound imaging system and the server improves workflow during the examination of the patient by avoiding display of sections of the worksheet in the graphical user interface that are not associated with the selected multiple choice option.

14. The method of claim 13 further comprising interacting with the server during the examination of the patient by sending patient information to the server and, in response, receiving from the server additional information for use by the sonographer.

15. The method of claim 14, wherein the patient information comprises at least one of a patient identifier and a study identifier.

16. The method of claim 14, wherein the patient information comprises at least one sonographer-flagged ultrasound image of the patient.

17. The method of claim 13 further comprising performing at least one of the following:
sending an instant message to a person remote from the medical diagnostic ultrasound imaging system; and
receiving an instant message from the person remote from the medical diagnostic ultrasound imaging system.

18. The method of claim 14, wherein the information for use by the sonographer is not specifically requested by the medical diagnostic ultrasound imaging system, and wherein the server is configured to determine what information for use by the sonographer to send based on the patient information received from the medical diagnostic ultrasound imaging system.

19. The method of claim 14, wherein the information for use by the sonographer is stored in a server-accessible storage location prior to the medical diagnostic ultrasound imaging system sending the patient information to the server.

20. The method of claim 14, wherein the information for use by the sonographer is generated by the server after the medical diagnostic ultrasound imaging system sends the patient information to the server.

21. The method of claim 13, wherein the worksheet and the ultrasound image of the patient are simultaneously displayed on one or more display devices of the medical diagnostic ultrasound imaging system.

22. The method of claim 13, wherein the method is performed by a controller that is integrated in the medical diagnostic ultrasound imaging system.

23-27. (canceled)

28. The medical diagnostic ultrasound imaging system of claim 1, wherein the server is external to the medical diagnostic ultrasound imaging system.

29. The medical diagnostic ultrasound imaging system of claim 1, wherein the server is internal to the medical diagnostic ultrasound imaging system.

30. The method of claim 13 further comprising receiving a response from a person remote from the medical diagnostic ultrasound imaging system for use by a sonographer during a remainder of the examination of the patient, wherein the response is based, at least in part, on the sonographer input in the worksheet, wherein the person remote from the medical diagnostic ultrasound imaging system and the medical diagnostic ultrasound imaging system are in different rooms.
